(19) United States
(12) Patent Application Publication
(10) Pub. No.: US 2014/0364654 A1
(43) Pub. Date: Dec. 11, 2014

(54) DIMETHYL ETHER (DME) PRODUCTION PROCESS

(71) Applicants: SARABJIT S. RANDHAVA, Evanston, IL (US); Richard L. Kao, Northbrook, IL (US); Todd L. Harvey, Palatine, IL (US); Bradley S. Novak, Skokie, IL (US); Jorge Romero Zabaleta, Mount Prospect, IL (US)

(72) Inventors: SARABJIT S. RANDHAVA, Evanston, IL (US); Richard L. Kao, Northbrook, IL (US); Todd L. Harvey, Palatine, IL (US); Bradley S. Novak, Skokie, IL (US); Jorge Romero Zabaleta, Mount Prospect, IL (US)

(73) Assignee: Unitel Technologies, Inc., Mount Prospect, IL (US)

(21) Appl. No.: 13/913,821

(22) Filed: Jun. 10, 2013

(51) Int. Cl. C07C 41/09

(52) U.S. Cl. CPC C07C 41/09
USPC 568/698

(57) ABSTRACT

Disclosed herein is a process for monetization of natural gas by producing fuel grade dimethyl ether (DME). The process includes three reactive stages with the first reactive stage being the conversion of natural gas into syngas, the second reactive stage being the conversion of syngas into crude methanol and the third reactive stage being the production of fuel grade dimethyl ether. The management and optimization of the water and steam circuits is important to maintain net overall system efficiency and mitigation of any liquid effluents.

### FIELD OF INVENTION

The present invention relates, generally, to a process for the production of fuel grade dimethyl ether (DME) from methanol dehydration via catalytic distillation. The methanol is produced from syngas in a methanol synthesis loop and this syngas is produced from natural gas in a steam reformer using either a pressurized burner or an atmospheric pressure burner.

### BACKGROUND OF INVENTION

Dimethyl ether (DME) is rapidly being recognized as the optimum energy vector for the 21st century. Its high oxygen content and absence of carbon to carbon bonds eliminate soot and particulates in the post combustion environment. The application of DME is especially logical in countries that are poor in oil and gas resources. DME is much more environmentally friendly than conventional hydrocarbon fuels.

DME's overall physical properties are similar to those of LPG. DME liquefies at 59 psia (6.1 bar) or −13° F. (−25° C.). Its vapor pressure at 122° F. (50° C.) is 170 psig (12.7 bar), while that of propane is 250 psig (18.3 bar). Since DME can readily exist in a liquid form, it is easily transportable in terms of international trade.
The DME end product, when it is utilized, will be 100% clean.
DME can be used as a one-to-one replacement as a fuel for diesel engines.
As a diesel fuel replacement, DME is 100% clean in terms of sulfur, 100% clean in terms of soot or particulates, and much cleaner than conventional fuels in terms of NO_{X} and CO₂ emissions.
DME is decomposed in a troposphere in less than a day; it does not cause ozone layer depletion.

DME can be produced from syngas (CO and H₂) generated by natural gas reforming directly as described by I K Hyun Kim et al. in REF. 1:

3CO+3H₂→CH₃OCH₃+CO₂ ΔH_{270° C.=-}258.73 KJ/mol (1)

Or from methanol synthesis and then methanol dehydration:

2CO+4H₂→2CH₃OH ΔH_{270° C.}=−201.84 KJ/mol (2)

2CH₃OH→CH₃OCH₃+H₂O ΔH_{270° C.}=−17.35 KJ/mol (3)

In the direct DME synthesis process when natural gas is used as the carbonaceous feedstock, it requires a H₂ to CO molar ratio to be close to 1.0 (EQ. 1) in the DME synthesis loop feed gas. Therefore, a huge amount of CO₂ is fed to the reformer to manipulate this ratio. The majority of added CO₂ then has to be removed by a solvent wash such as cold methanol (Rectisol™), or chilled Selexol™ physical solvent to avoid CO₂ build up in the DME synthesis loop. In addition, the CO₂ produced by EQ. 1 will also have to be removed at cryogenic condition, i.e. −40° F. (−40° C.) by the produced DME and some methanol (REF. 1). The DME and methanol produced are used here as the CO₂ absorption solvent. While the DME synthesis temperatures are 536 to 572° F. (260 to 300° C.), this causes a huge energy loss in heating and cooling. Because of these reasons, we abandon the natural gas to DME synthesis direct process in this invention.

In the indirect DME synthesis process, methanol will have to be synthesized first (EQ. 2) and then dehydrates the methanol synthesized to produce DME (EQ. 3). There are several methanol synthesis processes available. The major differences among these processes are in the methanol synthesis loop designs used to remove the heat generated by the highly exothermic methanol synthesis reactions. The method currently used by these processes is to increase the H₂ to CO molar ratio of the feed gas to the methanol synthesis loop far beyond the stoichiometric ratio in order to remove the exothermic heat. For instance, Imperial Chemical Industries (ICI) methanol synthesis process (REF. 2) uses a H₂/CO molar ratio in the feed gas to the methanol synthesis loop of 7.97; Johnson Matthey (REF. 3), 16.62; Exxon Mobil (REF. 4), 6.70; TEC (REF. 6) 10.53; and UNITEL (REF. 7), 7.05. In methanol synthesis, the feed gas to the methanol synthesis loop is characterized by the stoichiometric ratio (H₂—CO₂)/(CO+CO₂), often referred to as the module M. A module of 2.05 defines an ideal stoichiometric synthesis gas for formation of methanol. These high values of the H₂/CO molar ratio used by these methanol synthesis processes yield high module numbers also (Table 1).

TABLE 1
FEED SYNGAS COMPARISON WITH LITERATURE DATA IN METHANOL SYNTHESIS

| | Present | | | | | |
|---|---|---|---|---|---|---|
| | Invention | | | Exxon | | Johnson |
| Feed Gas | 1 | UNITEL | ICI | Mobil | TEC | Matthey |
| Phase | Vapor | Vapor | Vapor | Vapor | Vapor | Vapor |
| Temperature, ° C. (° F.) | 205 (401) | 110 (230) | 80 (176) | 77 (170) | 240 (464) | 230 (446) |
| Pressure, bar (psig) | 71 (1,015) | 82 (1,175) | 84 (1,204) | 85 (1,218) | 100 (1,436) | 85 (1,218) |

Feed Gas Comp., mol %

| | | | | | | |
|---|---|---|---|---|---|---|
| CH₄ | 10.68 | 5.74 | 9.33 | 12.05 | 1.35* | 10.10 |
| CO | 15.75 | 9.08 | 8.70 | 10.31 | 7.90 | 4.89 |
| CO₂ | 9.50 | 10.60 | 10.45 | 4.14 | 5.80 | 3.27 |
| H₂ | 61.16 | 64.00 | 69.37 | 69.03 | 83.20 | 81.24 |
| H₂O | 0.22 | 0.24 | 0.11 | 0.10 | 0.10 | 0.12 |
| N₂ | 2.27 | 9.76 | 1.66 | 3.84 | 1.35* | 0.00 |
| CH₄O | 0.42 | 0.58 | 0.38 | 0.53 | 0.30 | 0.38 |
| TOTAL | 100.00 | 100.00 | 100.00 | 100.00 | 100.00 | 100.00 |
| Feed Gas H₂ to CO | 3.88 | 7.05 | 7.97 | 6.70 | 10.53 | 16.62 |
| Molar Ratio | | | | | | |
| Feed Gas Module | 2.05 | 2.71 | 3.08 | 4.49 | 5.65 | 9.56 |
| Number | | | | | | |
| CO₂ in Feed Gas, wt % | 33.85 | 37.67 | 43.65 | 19.59 | 35.69 | 23.64 |
| Methanol Synthesis | 12.65 | 12.56 | 9.52 | 8.83 | — | 5.10 |
| Loop Recycle Gas MW | | | | | | |
| Methanol Synthesis | 1.24 | 2.35 | 1.99 | 4.00 | — | 3.00 |
| Loop Recycle to Make- | | | | | | |
| up Gas Molar Ratio | | | | | | |
| Methanol Synthesis | 29.65 | 15.00 | 5.64 | 1.48 | — | 8.85 |
| Loop Recycle Purge, % | | | | | | |
| Internal Reactor Cooling | No | Yes | Yes | No | Yes | Yes |
| H₂ Recovery from Purge | No | No | Yes | No | — | No |
| of Methanol Synthesis | | | | | | |
| Loop | | | | | | |
| Recovery from H₂ | Yes | Yes | Yes | No | — | No |
| Membrane Recycle Gas | | | | | | |

*Assume equal amount of CH₄ and N₂ in the gas mixture.

The drawback of these high module numbers is that they dilute the reactants which reduce the syngas conversion efficiency for methanol synthesis and meanwhile cause a tremendous increase in the energy required by the recycle stream compressor, in addition larger methanol synthesis reactor(s) and piping are also required. The details of this drawback will be further illustrated in Example 3.

It has now been found that the above drawback can be avoided by (i) purging recycle gas of the methanol synthesis loop to the H₂ membrane; (ii) recovering a H₂ rich stream from the H₂ membrane; (iii) purging recycle gas B (FIG. 1A) to the steam reformer HP burner; (iv) feeding both remaining recycle gas B and natural gas to the saturator; (v) manipulating these two purge rates to obtain 2.05 module number for the methanol synthesis feed gas and meanwhile also provide appropriate remaining recycle gas B flow to evaporate enough steam in the saturator for the downstream steam reforming reactions.

### SUMMARY OF THE INVENTION

It is the object of the present invention to provide a process of economically and efficiently producing DME, which comprises converting the natural gas into syngas by a pressurized reformer, which then undergoes methanol synthesis and catalytic distillation dehydration to convert raw methanol into fuel grade DME.

In order to accomplish the above object, the present invention provides a process for the production of DME comprising the following steps of:
Purging a portion of recycle gas B (FIG. 1A) from the H₂ membrane to the steam reformer HP burner;
Simultaneously subjecting a feedstock mixture including natural gas and the remaining of the recycle gas B to the bottom of a saturator;
Feeding a hot water stream to the top of the saturator and allowing the hot water to evaporate in the presence of the rising gaseous stream as it travels down the saturator. In this way, all the high pressure steam required for the downsteam reforming reactions is provided;
Steam reforming the saturated natural gas and the remaining recycle gas B to produce a syngas;
Recovering the heat from the reformer effluent by superheating the saturated high pressure steam to generate electric power in a syngas heat recovery boiler and superheating boiler feed water to generate superheated medium pressure steam for additional electric power generation;
Directing the effluent from the medium pressure heat recovery boiler into a cooler where bulk of the water vapor in the syngas is condensed and knocked-out;
Combining the compressed syngas with the methanol synthesis loop recycle gas A (FIG. 1A) to yield a module number of 2.05 which is the ideal module number for methanol synthesis;
Subjecting the combined gas mixture to the methanol synthesis loop in the presence of Haldor Topsoe MK-121 methanol synthesis catalyst to obtain a reaction product gas mixture including methanol, carbon dioxide, water vapor, inerts like methane and nitrogen, and unconverted hydrogen and carbon monoxide;
Condensing the reaction product gas mixture to separate the methanol and the water produced;
Reducing the pressure of the crude methanol product to evaporate dissolved gases;
Purifying the low pressure crude methanol product by a light end distillation column to strip more dissolved gases;
Pumping the purified crude methanol product to a pressure of about 115 psig (9 bar) and then it is fed to a catalytic distillation dehydration column for the production of fuel grade DME.

### BRIEF DESCRIPTION OF THE DRAWINGS

FIG. 1A is a simplified process flow diagram for the production of fuel grade DME from natural gas.

FIG. 1B is the complete turboexpander-turbocompressor system.

FIG. 2 is a simplified material balance for the natural gas to DME via the methanol dehydration route using three adiabatic methanol synthesis reactors in series.

FIG. 3 is a steam/water balance for the process of natural gas to DME via the methanol dehydration route using three adiabatic methanol synthesis reactors in series.

FIG. 4 is the operation conditions of the turboexpander-turbocompressor system.

FIG. 5 is a simplified process flow diagram for the natural gas to DME via the methanol dehydration route using a single MRF methanol synthesis reactor.

### DETAILED DESCRIPTION OF THE INVENTION

For illustration purposes, a methanol synthesis loop with three adiabatic fixed bed reactors in series 8 with internal cooling between the reactors had been chosen for Example 1; and a steam-rising Multi-stage indirect cooling and Radial Flow (MRF) single methanol synthesis catalytic reactor 8 has been chosen for Example 3.

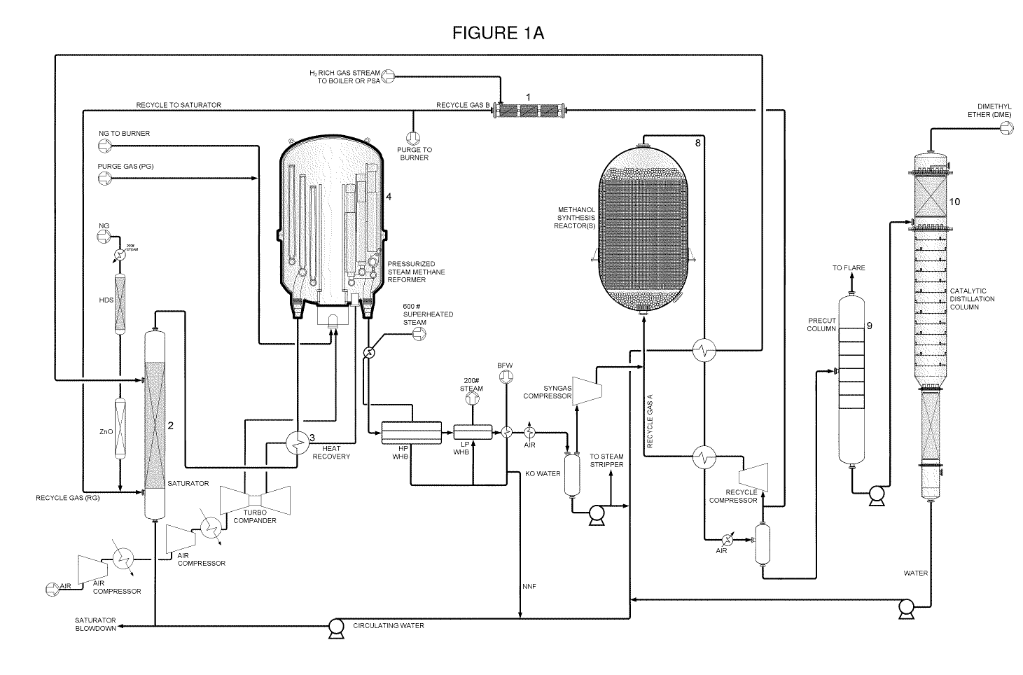

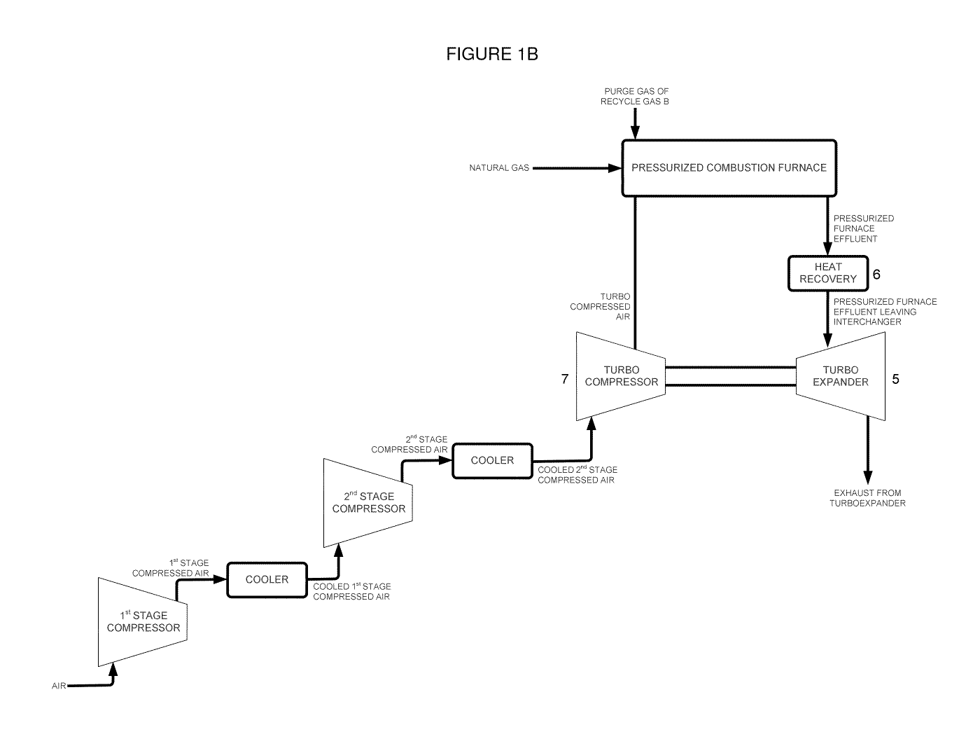

A pressurized gaseous stream of desulfurized NG and the majority of the recycle gas B from the H₂ membrane System 1 (FIG. 1A) is fed to the bottom of a saturator 2 while one liquid stream of hot water under pressure is fed at the top of the saturator 2. The hot water is allowed to evaporate in the presence of the rising gaseous stream as it travels down the saturator 2. In this way, 100% of the high pressure steam required for the downstream steam reforming reactions can be provided, which would otherwise have been supplied through high energy consumption.

The saturated natural gas and the majority of the recycle gas B stream then is preheated by the burner flue gas waste heat recovery section 3 before entering the tubular steam reformer 4 operated at 1,600° F. (871° C.) and 300 psig (21.7 bar). One method of overcoming problems of stress-rupture failures of the reformer catalyst tubes due to high temperature and high pressure operation is to use a pressurized burner in the reformer which is called pressurized reformer. Burner pressures are suitably maintained at about 100 to 250 psig (7.9 to 18.3 bar) and preferably about 150 to 200 psig (11.4 to 14.8 bar). The saturated natural gas and the majority of the recycle gas B mixture is brought to the requisite elevated temperature and supplied the endothermic heat for the steam reforming reactions by transfer of heat from the hot burner effluent gas through the metal walls of catalyst tubes. The pressurized reformer 4 is different to a conventional reformer in that the primary heat transfer mechanism is convection rather than radiation. The integrated internal heat recovery design of the pressurized reformer 4 ensures an improved fuel demand to meet reforming heat load requirements and improved overall energy efficiency. One way to compare the reformer overall energy efficiency is by the comparison of exit temperatures of reformer process gases and flue gases (Table 2). Another advantage of the pressurized reformer 4 is that it is less than a quarter of the weight and size of a conventional reformer. The uniformity of the reaction and combustion conditions of the pressurized reformer 4 avoid undesirable carbon formation and give very efficient combustion with minimum excess air for the fuel combustion, avoiding unwanted heat losses and resulting in a lower fuel consumption for a given reformer duty.

TABLE 2
STEAM REFORMER COMPARISON

| CONVENTIONAL | PRESSURIZED |
|---|---|
| Has to be field constructed | Shop fabrication to enable a high |
| and assembled | level of quality control & reduction |
| | in project construction schedules |
| Non-transportable | Truck transportable dimensions |
| Thermally inefficient, heat | The primary heat transfer mechanism is |
| transfer is radiative | convective |
| Reformer process gas exit | Reformer process gas exit temperature: |
| temperature: about 1,600° F. | about 1,020 to 1,050° F. (549 to |
| (871° C.) | 566° C.) |
| Reformer flue gas exit | Reformer flue gas exit temperatures: |
| temperatures: 1,825 to 1,900° | 1,060 to 1,100° F. (571 to 593° C.) |
| F. (996 to 1,038° C.) | |
| Fuel consumption: 100% | Fuel consumption: 46% |
| Size of weight of reformer: | Size & weight of reformer: less than 25% |
| 100% | |
| Reformer duty: 100% | Reformer duty: 75% |
| Reformer flue gas exit flow | Reformer flue gas exit flow rate: 3% |
| rate: 100% | |

A turboexpander 5 (FIG. 1B) is placed at the end of the burner flue gas waste heat recovery section 6 to recover waste energy by driving the last stage of a three-stage air compressor 7. It helps cut the air compression energy needs by more than 40%.

A low or atmospheric pressure burner can also be used in the reformer which is called conventional reformer, and by doing so the primary heat transfer mechanism will be radiation rather than convection. The reformer process gas effluent temperature will be about 1,600° F. (871° C.) instead of about 1,020 to 1,050° F. (549 to 566° C.) and the reformer flue gas effluent temperature will be 1,825° F. (996° C.) minimum, 1,900° F. (1,038° C.) maximum instead of about 1,060 to 1,100° F. (571 to 593° C.) as shown in Table 2. Now special condition is required in design to overcome problems of stress-rupture failures of the reformer catalyst tubes due to high temperature and high pressure operations.

The sensible heat of the hot syngas produced by the pressurized reformer 4 is recovered by superheating a high pressure saturated steam for electric power generation and then superheating a medium pressure boiler feed water for additional electric power generation. This syngas is then further cooled to knockout water before it is compressed to methanol synthesis pressure (1,045 psig or 73 bar). At this point, the conventional methanol synthesis catalyst usually requires an acid gas (CO₂ and sulfur compounds) removal step to lower the CO₂ content in the syngas to be less than about 3 mol % in order to maintain the catalyst activity when natural gas is used as the carbonaceous fuel in the steam reformer and a module number of 2.05 is desired for the feed gas to the methanol synthesis loop. A solvent wash by amines, Selexol™ Rectisol™, etc. is needed. However, a high capital cost and high energy consumption are associated to pump the solvent around and to regenerate the solvent. Recently, a breakthrough of methanol synthesis catalyst named MK-121 was developed by Haldor Topsoe. MK-121 ensures very high conversion efficiency whether the synthesis gas is rich in carbon dioxide, carbon monoxide or both. Furthermore, MK-121 allows operation at lower temperatures than conventional methanol synthesis catalysts where conditions for byproduct formation is less favorable. MK-121 also has a high capacity for sulfur uptake and metal carbonyls and can in most cases, completely guard itself against residual poisons. Thus, the costly acid gas removal step before the methanol synthesis loop is eliminated permanently.

The compressed syngas sometimes called make-up syngas, is mixed with the methanol synthesis loop recycle gas A, preheated by the process gas from the last adiabatic methanol synthesis reactor 8 before it is fed to the methanol synthesis loop. The mixed methanol synthesis feed gas is characterized by the stoichiometric ratio (H₂—CO₂)/(CO+CO₂), often referred to as the module M as discussed above. A module of 2 defines a stoichiometric synthesis gas for formation of methanol. In actual cases, a slightly higher module number like 2.05 will be used. Other important properties of the synthesis gas are the CO to CO₂ molar ratio and the concentration of inerts. A high CO to CO₂ molar ratio will increase the reaction rate and the achievable per pass conversion. In addition, the formation of water will decrease, which reduces the catalyst deactivation rate. High concentration of inerts will lower the partial pressure of the active reactants. Inerts in the methanol synthesis are typically methane and nitrogen which are controlled by the purge rates from the methanol synthesis loop and from recycle gas B.

In the methanol synthesis loop, conversion of syngas into crude methanol takes place. Crude methanol is a mixture of methanol, a small amount of water, dissolved gases, and traces of byproducts. The conversion of hydrogen and carbon oxides to methanol is described by the following reactions:

CO+2H₂→CH₃OH ΔH_{270° C.}=−100.92 KJ/mol (4)

CO₂+3H₂→CH₃OH+H₂O ΔH_{270° C.}=−61.38 KJ/mol (5)

CO+H₂O→CO₂+H₂ ΔH_{270° C.}=−39.54 KJ/mol (6)

The methanol synthesis is exothermic and the maximum conversion is obtained at low temperature and high pressure. A challenge in the design of methanol synthesis is to remove the heat of reaction efficiently and economically. Today, six different designs of methanol synthesis reactors are commercially in operation: (1) quench reactor; (2) adiabatic reactors in series; (3) tube cooling reactor; (4) steam rising isothermal tubular bed reactor; (5) steam rising isothermal boiler coil reactor; (6) steam rising Multi-stage indirect-cooling and Radial Flow (MRF) Reactor.

In our invention, about 90 to 95% of the methanol produced is by EQ. 4, and only 5 to 10% is by EQ. 5. Another important characteristic of our invention is that a high purge rate, about 30%, is applied to the methanol synthesis loop using three adiabatic reactors in series (Example 1) and about 80% in Example 3 when a single MRF reactor is used in the methanol synthesis loop. The majority of the recycle gas B after the H₂ membrane 1 (H₂ removal step) is recycled to pick up steam in the saturator 2 and to supply the CO₂ needed for the reformer 4 to manipulate the module number for permitting optimization of the syngas composition for methanol production. The H₂ rich stream removed from the H₂ membrane 1 can either go through a PSA system to produce pure H₂ at 260 psig (19 bar) in Example 1, and at 400 psig (28.6 bar) in Example 3, or can be used as boiler fuel for the electric power/steam generation.

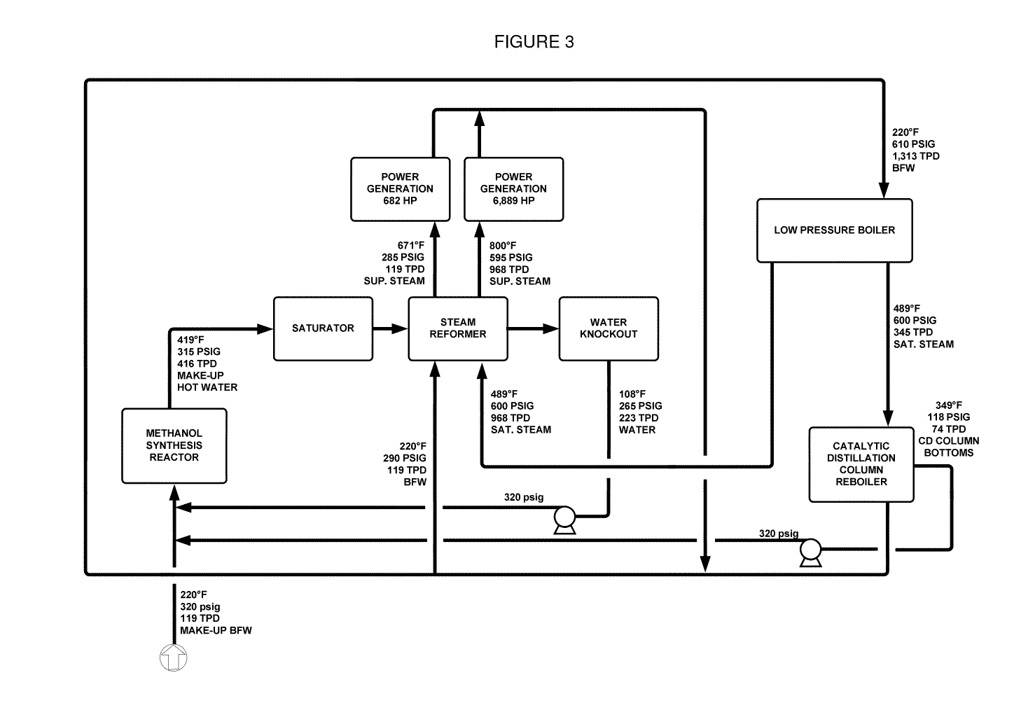

The process gas stream from the last adiabatic methanol synthesis reactor 8 is used to preheat the feed gas to the first reactor before it is cooled further to condense the crude methanol product. The crude methanol stream is let down in pressure from methanol synthesis pressure to about 10 psig (1.7 bar) in order to evaporate dissolved gases and then is fed to a light end distillation column 9 to strip more dissolved gases. The purified crude methanol now containing mainly methanol and water is pumped to a pressure of about 115 psig (9 bar) and is fed to a catalytic distillation dehydration column 10 for the production of fuel grade DME. The water produced from the catalytic distillation dehydration column bottom is combined with the knockout water and make-up boiler feed water and heat exchanged with the internal methanol synthesis reactor effluents before it is fed to the top of the saturator 2 (FIG. 3).

Although the invention has been described with reference to its various embodiments, from this description, those skilled in the art may appreciate changes and modifications thereto, which do not depart from the scope and spirit of the invention as described herein and claimed hereafter. The following examples illustrate specific embodiments of the invention, and is not meant to limit the scope of the invention in any way.

### Example 1

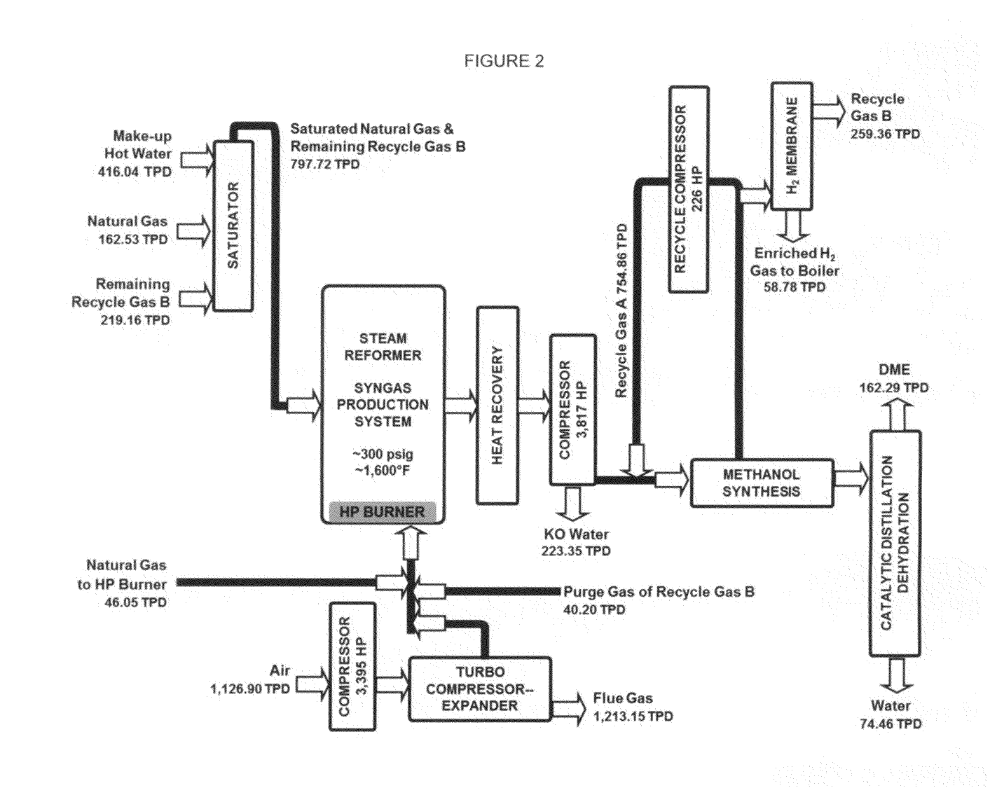

A combined gaseous mixture of 804.78 lbmol/hr of natural gas and 762.92 lbmol/hr of recycle gas B are fed to the bottom of a saturator, while a stream of hot water is fed at the top of the saturator (FIG. 2). The rising gaseous stream evaporates the hot water as it travels down the saturator. The flow rate of the recycle gas B stream and the CO₂ concentration in the stream are manipulated to obtain 2.05 module number for the methanol synthesis feed gas and meanwhile also to evaporate enough steam in the saturator for the downstream steam reforming reactors.

The saturated natural gas and the remaining recycle gas B mixture is then preheated by the HP burner flue gas before entering the tubular steam reformer operated at 1,600° F. (871° C.) and 300 psig (21.7 bar). A syngas with the composition below is obtained (Table 3):

TABLE 3
SYNGAS FROM PRESSURIZED STEAM REFORMER

| PHASE | VAPOR |
|---|---|
| Temp., ° F. (° C.) | 1,021.0 (544.4) |
| Pressure, psig (bar) | 295 (21.4) |
| Flowrate, lbmol/hr | 5,030.10 |
| H₂/CO molar ratio | 3.0395 |
| Composition | Mol % |
| CH₄ | 5.69 |
| CO₂ | 5.90 |
| N₂ | 1.20 |
| H₂O | 20.91 |
| CO | 16.41 |
| H₂ | 49.88 |

The sensible heat of the hot syngas produced by the pressurized reformer is recovered first by superheating a high pressure stream of saturated steam at 600 psig (42.4 bar) and 489° F. (253.9° C.) to 800° F. (426.7° C.) which generates 6889 hp electric power through a steam turbine, and then superheats a medium pressure boiler feed water at 290 psig (21.0 bar) and 220° F. (104.4° C.) to 671° F. (355.0° C.) which then generates an additional 682 hp electric power. The syngas is then further cooled to knockout most of its moisture content, 1,032.97 lbmol/hr before it is compressed to the methanol synthesis pressure, 1,045 psig (73.1 bar). This compressed syngas is sometimes called make-up syngas.

The make-up syngas is mixed with the methanol synthesis loop recycle gas A to obtain a methanol synthesis loop feed gas with an appropriate module number by methods as discussed above. For illustration purposes, a synthesis loop with three adiabatic fixed bed reactors in series with internal cooling between the reactors is chosen. The cooling is provided by preheat of boiler feed water or generation of medium pressure steam. The combined gas mixture is preheated by the process gas from the last adiabatic methanol synthesis reactor to 401° F. (205° C.) before it is fed to the methanol synthesis loop. A 30% purge gas rate is applied to the methanol synthesis loop and 85% of the recycle gas B is fed to the bottom of the saturator to pick up enough steam in the saturator and meanwhile to get a module number of 2.05 for the methanol synthesis feed gas. The methanol synthesis loop feed gas has the following composition (Table 4):

TABLE 4
METHANOL SYNTHESIS LOOP FEED GAS

| PHASE | VAPOR |
|---|---|
| Temp., ° F. (° C.) | 401.0 (205.0) |
| Pressure, psig (bar) | 1,018.5 (71.2) |
| Flowrate, lbmol/hr | 8,971.74 |
| H₂/CO molar ratio | 3.8847 |
| Module | 2.05 |
| Composition | Mol % |
| CH₄ | 10.68 |
| CO₂ | 9.50 |
| N₂ | 2.27 |
| H₂O | 0.22 |
| CO | 15.75 |
| H₂ | 61.16 |
| CH₄O | 0.42 |

The process gas stream from the last adiabatic methanol synthesis reactor is used to preheat the feed gas before it is cooled further to 105° F. (40.6° C.) to condense the crude methanol product which has the following composition (Table 5):

TABLE 5
CRUDE METHANOL PRODUCT

| PHASE | VAPOR |
|---|---|
| Temp., ° F. (° C.) | 105.0 (40.6) |
| Pressure, psig (bar) | 974.5 (68.2) |
| Flowrate, lbmol/hr | 680.46 |
| Composition | Mol % |
| CH₄ | 0.46 |
| CO₂ | 4.42 |
| N₂ | 0.02 |
| H₂O | 7.47 |
| CO | 0.07 |
| H₂ | 0.22 |
| CH₄O | 87.34 |
| Acetic Acid | 13.81 ppm |
| Acetone | 13.28 ppm |
| Ethanol | 29.56 ppm |

This crude methanol stream is let down in pressure from 974 psig (68.2 bar) to 10 psig (1.7 bar) to evaporate dissolved gases and then is fed to a 15 stage light end distillation column to strip more dissolved gases. By letting down the pressure to 10 psig (1.7 bar) instead of 120 psig (9.3 bar), it saves 82% of the condenser cooling duty and 65% of the reboiler heat duty for the light end distillation column (Table 6).

TABLE 6
LIGHT END DISTILLATION COLUMN COMPARISON

| Cases | Case 1 | Case 2 |
|---|---|---|
| Pressure, psig (bar) | 120 (9.3) | 10 (1.7) |
| Stages | 15 | 15 |
| Molar Reflux Ratio | 2 | 2 |
| Condenser Duty, Btu/hr | −1,370,906 | −248,659 |
| Reboiler Duty, Btu/hr | 4,264,289 | 1,477,999 |

The bottom stream from the light end distillation column contains mainly methanol and water (Table 7).

TABLE 7
PURIFIED CRUDE METHANOL PRODUCT

| PHASE | VAPOR |
|---|---|
| Temp., ° F. (° C.) | 177.2 (80.7) |
| Pressure, psig (bar) | 11.0 (1.8) |
| Flowrate, lbmol/hr | 637.88 |
| Composition | Mol % |
| CH₄ | 0.00 |
| CO₂ | 0.00 |
| N₂ | 0.00 |
| H₂O | 7.95 |
| CO | 0.00 |
| H₂ | 0.00 |
| CH₄O | 92.05 |
| Acetic Acid | 14.73 ppm |
| Acetone | 13.09 ppm |
| Ethanol | 31.33 ppm |

The bottom stream is pumped to 116 psig (9 bar) and is then fed to a 30 stage catalytic distillation dehydration column (Table 8) for the production of 293.58 lbmol/hr or 162.29 ton/day of fuel grade DME.

TABLE 8
CATALYTIC DISTILLATION DEHYDRATION COLUMN
Feed Stream

| Phase | Liquid | |
|---|---|---|
| Temp., ° F. (° C.) | 177.4 | (80.8) |
| Pressure, psig (bar) | 116.4 | (9) |
| Flowrate, lbmol/hr | 637.88 | |
| Composition | Mol % | |
| CH₄O | 92.05 | |
| H₂O | 7.95 | |
| Acetic Acid | 14.73 | ppm |
| Acetone | 13.09 | ppm |
| Ethanol | 31.33 | ppm |

Catalytic Distillation Column

| Stripping Stages | 21 to 30 | |
|---|---|---|
| Total Stages | 30 | |
| Rectification Stages | 1 to 7 | |
| Reaction Stages | 8 to 20 | |
| Feed Stage | 8 | |
| Column Pressure, psig (bar) | 116 (9) | |
| Molar Reflux Ratio | 9 | |
| Distillate to CH₄O Feed Ratio | 0.5 | |
| DME Purity | 99.9834 | mol % |
| | 99.9884 | wt % |

The H₂ rich stream removed from the H₂ membrane can either go through a PSA system to produce 7.70 MMSCFD of pure hydrogen at 260 psig (19 bar) or can be used as boiler fuel to produce 345 ton/day of 600 psig saturated steam for the catalytic distillation dehydration column reboiler and 6,889 HP of electric power which is about 98% of the power requirements for the entire DME plant.

The water stream produced at the catalytic distillation dehydration column bottom is 99.97 mol % or 99.94 wt % pure and there is no need for any waste water treatment. It is combined with the knockout water and make-up boiler feed water, heat exchanged with the internal methanol synthesis reactor effluents before it is fed to the top of the saturator (FIG. 3).

### Example 2

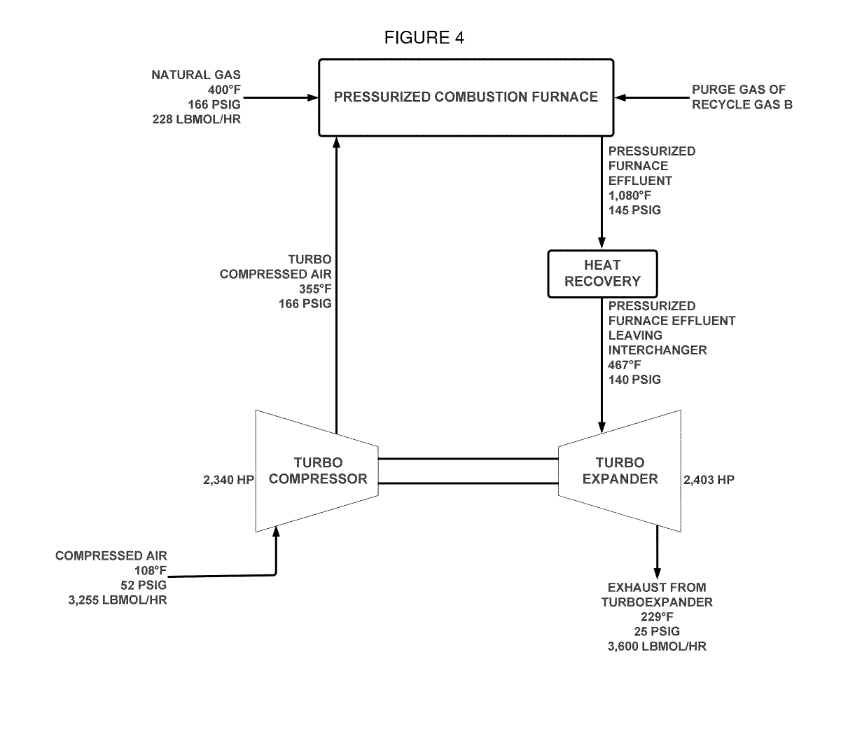

The pressurized furnace effluent leaving the interchanger at 467° F. (241.7° C.) and 140 psig (10.7 bar) is directed to a turboexpander to recover the waste energy by driving a turbocompressor to compress air from 52.3 psig (4.6 bar) to 166.3 psig (12.5 bar) which accounts for 41% of total air compression energy (FIG. 4).

Kunio Hirotani et al. (REF. 6) disclosed an optimum catalytic reactor design for methanol synthesis called steam rising Multi-stage indirect cooling and Radial Flow (MRF) single methanol synthesis catalytic reactor, in which the heat of the highly exothermic methanol synthesis reactions over the catalyst bed is removed by means of cooling tubes arranged adequately in the bed. Due to the large cross surface area for syngas flow in a radial flow pattern, extremely small pressure drop through the catalyst bed is resulted and an ideal temperature profile is accomplished for achieving higher conversion of syngas per pass on the same volume of catalyst.

The specification of a 5,000 ton/day MRF reactor: Inlet and outlet gas compositions, operating conditions are summarized in Table 9. The last column in Table 9 is the simulated outlet gas composition by Aspen Plus Basic Engineering V7.3.

TABLE 9
SPECIFICATION OF A 5,000 TON/DAY MRF REACTOR

| Composition, | Inlet | Outlet | Simulated Out- |
|---|---|---|---|
| mol % | Gas | Gas | let Gas |
| H₂ | 83.2 | 77.3 | 77.3 |
| CO | 7.9 | 2.1 | 2.2 |
| CO₂ | 5.8 | 4.4 | 4.4 |
| CH₄ + N₂ | 2.7 | 3.2 | 3.2 |
| H₂O | 0.1 | 2.8 | 2.7 |
| CH₄O | 0.3 | 10.2 | 10.2 |
| Total | 100.0 | 100.0 | 100.0 |
| Temperature, ° C. (° F.) | 240 (464) | 260 (500) | 260 (500) |
| Pressure, bar (psig) | 100 (1,436) | 99 (1,421) | 99 (1,421) |

### Example 3

In this example, the natural gas feed rate and conditions are the same as in Example 1 except that the three adiabatic methanol synthesis reactors in series are replaced by the above single MRF reactor. Due to the higher conversion of the syngas (mainly CO conversion) to methanol is achieved in the MRF reactor, a higher methanol synthesis loop recycle purge about 80% and about 5% purge of the H₂ depleted H₂ membrane recycle gas B are required to yield the ideal feed gas module number of 2.05 to the methanol synthesis loop.

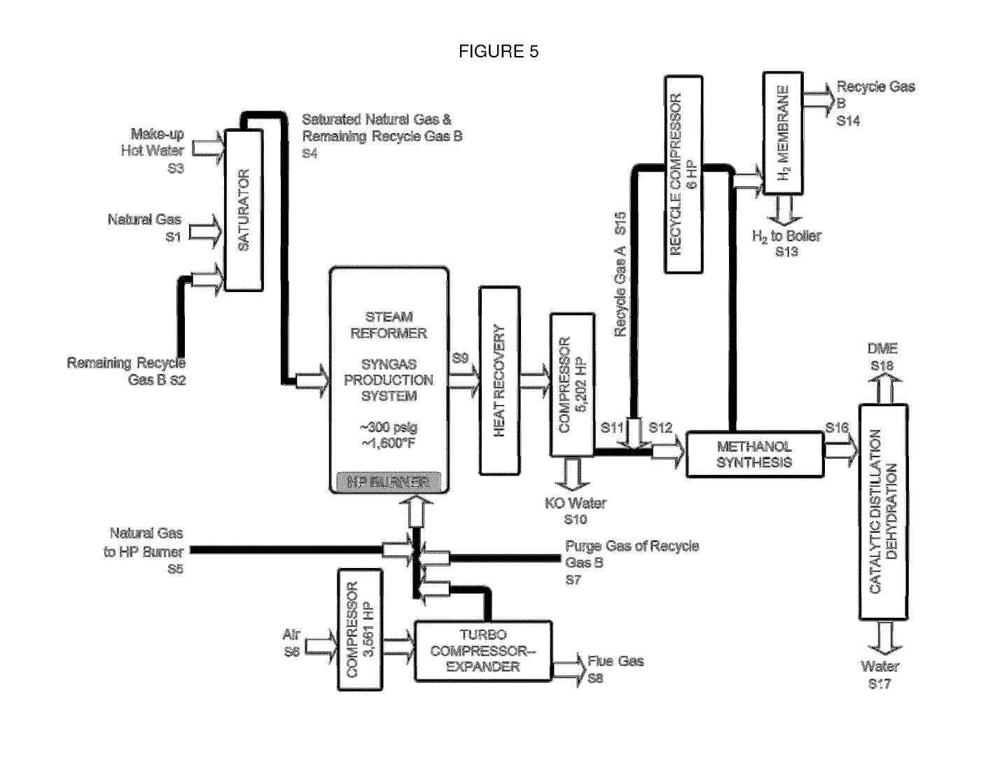

In the following Table 10, the flow rates, temperatures, pressures, enthalpy, vapor fractions and component mole fractions, etc. of all the streams shown in FIG. 5 are presented. In this example, the feed gas flow rate to the methanol synthesis loop reduces from 8,971.74 lbmol/hr to 4,694.32 lbmol/hr which is 47.7% smaller. This means that for the same amount of natural gas feed rate only about half the reactor volume and catalyst are required. It is amazed to find out that even with the 47.7% smaller methanol synthesis reactor, the DME production from the same natural gas feed rate as used in Example 1 has increased from 162.29 tons/day to 178.95 tons/day.

TABLE 10
SIMPLIFIED MATERIAL BALANCE FOR THE NATURAL GAS TO DME VIA THE METHANOL DEHYDRATION ROUTE USING A SINGLE MRF METHANOL SYNTHESIS REACTOR

[symbol]

| | | Stream No. | | | | | | | | |
|---|---|---|---|---|---|---|---|---|---|---|
| | | 1 | 2 | 3 | 4 | 5 | 6 | 7 | 8 | 9 |
| | | Stream Name | | | | | | | | |
| | | | | | Saturated Natural | | | | Flue Gas | |
| | | | Remaining | | Gas & Remaining | | | Purge Gas of | from Turbo | |
| | | Natural Gas | Recycle Gas B | Hot Water | Recycle Gas B | Natural Gas | Air to | Recycle Gas B | Compressor | Syngas from |
| | | to Saturator | to Saturator | to Saturator | to Reformer | to HP Burner | Compressor | to HP Burner | Expander | Reformer |
| Total Flow lbmol/hr | | 804.78 | 868.93 | 1,973.22 | 3,660.32 | 284.05 | 3,415.00 | 45.73 | 3,743.26 | 5,239.86 |
| Total Flow lb/hr | | 13,543.69 | 20,928.35 | 35,548.00 | 70,019.84 | 4,780.24 | 98,524.11 | 1,101.49 | 104,406.00 | 70,019.84 |
| Total Flow cuft/hr | | 22,250 | 24,109 | 672 | 94,546 | 14,524 | 1,360,440 | 1,445 | 686,879 | 270,307 |
| Temperature ° F. | | 400 | 400 | 420 | 370 | 400 | 86 | 79 | 225 | 1021 |
| Pressure, psia | | 335 | 335 | 330 | 330 | 181 | 15 | 181 | 40 | 310 |
| Vapor Fraction | | 1 | 1 | 0 | 1 | 1 | 1 | 1 | 1 | 1 |
| Liquid Fraction | | 0.00 | 0.00 | 1.00 | 0.00 | 0.00 | 0.00 | 0.00 | 0.00 | 0.00 |
| Average Mole Weight | | 16.83 | 24.09 | 18.02 | 19.13 | 16.83 | 28.85 | 24.09 | 27.89 | 13.36 |
| Density lbmol/cuft | | 0.04 | 0.04 | 2.94 | 0.04 | 0.02 | 0.00 | 0.03 | 0.01 | 0.02 |
| Density lb/cuft | | 0.61 | 0.87 | 52.94 | 0.74 | 0.33 | 0.07 | 0.76 | 0.15 | 0.26 |
| Mole Frac | | | | | | | | | | |
| Methane, CH4 | 16.04 | 0.9520 | 0.3015 | 0.0000 | 0.2805 | 0.9520 | 0.0000 | 0.3015 | 0.0000 | 0.0542 |
| Carbon Dioxide, CO2 | 44.01 | 0.0070 | 0.1939 | 0.0000 | 0.0468 | 0.0070 | 0.0000 | 0.1939 | 0.0854 | 0.0572 |
| Nitrogen, N2 | 28.01 | 0.0130 | 0.1569 | 0.0000 | 0.0401 | 0.0130 | 0.7900 | 0.1569 | 0.7236 | 0.0280 |
| Oxygen, O2 | 32.00 | 0.0000 | 0.0000 | 0.0000 | 0.0000 | 0.0000 | 0.2100 | 0.0000 | 0.0300 | 0.0000 |
| Water, H2O | 18.02 | 0.0000 | 0.0000 | 1.0000 | 0.5473 | 0.0000 | 0.0000 | 0.0000 | 0.1602 | 0.2070 |
| Carbon Monoxide, CO | 28.01 | 0.0000 | 0.2001 | 0.0000 | 0.0475 | 0.0000 | 0.0000 | 0.2001 | 0.0005 | 0.1593 |
| Hydrogen, H2 | 2.02 | 0.0000 | 0.1337 | 0.0000 | 0.0317 | 0.0000 | 0.0000 | 0.1337 | 0.0002 | 0.4941 |
| Methanol, CH4O | 32.04 | 0.0000 | 0.0139 | 0.0000 | 0.0000 | 0.0000 | 0.0000 | 0.0139 | 0.0000 | 0.0000 |
| DME, C2H6O-1 | 46.07 | 0.0000 | 0.0000 | 0.0000 | 0.0000 | 0.0000 | 0.0000 | 0.0000 | 0.0000 | 0.0000 |
| Ethane, C2H6 | 30.07 | 0.0250 | 0.0000 | 0.0000 | 0.0055 | 0.0250 | 0.0000 | 0.0000 | 0.0000 | 0.0000 |
| Propane, C3H8 | 44.10 | 0.0030 | 0.0000 | 0.0000 | 0.0007 | 0.0030 | 0.0000 | 0.0000 | 0.0000 | 0.0000 |
| Acetic Acid, C2H4O-01 | 60.05 | 0.0000 | 0.0000 | 0.0000 | 0.0000 | 0.0000 | 0.0000 | 0.0000 | 0.0000 | 0.0000 |
| Acetone, C3H6O-01 | 58.08 | 0.0000 | 0.0000 | 0.0000 | 0.0000 | 0.0000 | 0.0000 | 0.0000 | 0.0000 | 0.0000 |
| Ethanol, C2H6O-02 | 46.07 | 0.0000 | 0.0000 | 0.0000 | 0.0000 | 0.0000 | 0.0000 | 0.0000 | 0.0000 | 0.0000 |
| Butenol, C4H10-01 | 74.12 | 0.0000 | 0.0000 | 0.0000 | 0.0000 | 0.0000 | 0.0000 | 0.0000 | 0.0000 | 0.0000 |
| Enthalpy Btu/lbmol | | −29638.68 | −50499.93 | −116550.00 | −74048.62 | −29611.36 | 60.02 | −53229.89 | −30050.45 | −33087.79 |
| Enthalpy Btu/lb | | −1761.17 | −2096.72 | −6469.47 | −3870.93 | −1759.55 | 2.08 | −2210.07 | −1077.40 | −2476.09 |
| Enthalpy MMBtu/hr | | −23853000.00 | −43881000.00 | −233470000.00 | −271040000.00 | −8411100.00 | 204984.00 | −2434400.00 | −112490000.00 | −173380000.00 |
| Entropy Btu/lbmol-R | | −20.81 | −0.95 | −29.93 | −10.66 | −19.55 | 1.13 | −3.68 | −0.16 | 4.79 |
| Entropy Btu/lb-R | | −1.24 | −0.04 | −1.66 | −0.56 | −1.16 | 0.04 | −0.15 | −0.01 | 0.36 |

| | | Stream No. | | | | | | | | |
|---|---|---|---|---|---|---|---|---|---|---|
| | | 10 | 11 | 12 | 13 | 14 | 15 | 16 | 17 | 18 |
| | | Stream Name | | | | | | | | |
| | | | Make-up Syngas | Feed Gas to | | | | Raw Methanol to | | |
| | | Knockout | to Methanol | Methanol | Hydrogen | Recycle | Recycle | Catalytic Distillation | | DME |
| | | Water | Synthesis Loop | Synthesis Loop | to Boiler | Gas B | Gas A | Dehydration | Wastewater | Product |
| Total Flow lbmol/hr | | 1,065.28 | 4,174.59 | 4,694.34 | 1,164.35 | 914.66 | 519.75 | 689.19 | 365.47 | 323.71 |
| Total Flow lb/hr | | 19,194.39 | 50,825.45 | 57,537.95 | 4,819.97 | 22,029.85 | 6,712.46 | 21,498.72 | 6,586.25 | 14,912.47 |
| Total Flow cuft/hr | | 311 | 23,730 | 33,533 | 17,075 | 3,901 | 2,247 | 466 | 118 | 380 |
| Temperature ° F. | | 108 | 275 | 464 | 125 | 125 | 108 | 177 | 349 | 105 |
| Pressure, psia | | 280 | 1455 | 1450 | 435 | 1420 | 1455 | 131 | 133 | 131 |
| Vapor Fraction | | 0 | 1 | 1 | 1 | 1 | 1 | 0 | 0 | 0 |
| Liquid Fraction | | 1.00 | 0.00 | 0.00 | 0.00 | 0.00 | 0.00 | 1.00 | 1.00 | 1.00 |
| Average Mole Weight | | 18.02 | 12.17 | 12.26 | 4.14 | 24.09 | 12.91 | 31.19 | 18.02 | 46.07 |
| Density lbmol/cuft | | 3.42 | 0.18 | 0.14 | 0.07 | 0.23 | 0.23 | 1.48 | 3.09 | 0.85 |
| Density lb/cuft | | 61.65 | 2.14 | 1.72 | 0.28 | 5.65 | 2.99 | 46.12 | 55.74 | 39.29 |
| Mole Frac | | | | | | | | | | |
| Methane, CH4 | 16.04 | 0.0000 | 0.0681 | 0.0754 | 0.0029 | 0.3015 | 0.1343 | 0.0000 | 0.0000 | 0.0000 |
| Carbon Dioxide, CO2 | 44.01 | 0.0001 | 0.0718 | 0.0762 | 0.0462 | 0.1939 | 0.1112 | 0.0000 | 0.0000 | 0.0000 |
| Nitrogen, N2 | 28.01 | 0.0000 | 0.0352 | 0.0390 | 0.0023 | 0.1569 | 0.0703 | 0.0000 | 0.0000 | 0.0000 |
| Oxygen, O2 | 32.00 | 0.0000 | 0.0000 | 0.0000 | 0.0000 | 0.0000 | 0.0000 | 0.0000 | 0.0000 | 0.0000 |
| Water, H2O | 18.02 | 0.9999 | 0.0047 | 0.0042 | 0.0002 | 0.0000 | 0.0001 | 0.0605 | 0.9997 | 0.0000 |
| Carbon Monoxide, CO | 28.01 | 0.0000 | 0.2000 | 0.1878 | 0.0027 | 0.2001 | 0.0895 | 0.0000 | 0.0000 | 0.0000 |
| Hydrogen, H2 | 2.02 | 0.0000 | 0.6202 | 0.6167 | 0.9453 | 0.1337 | 0.5883 | 0.0000 | 0.0000 | 0.0000 |
| Methanol, CH4O | 32.04 | 0.0000 | 0.0000 | 0.0007 | 0.0003 | 0.0139 | 0.0063 | 0.9394 | 0.0002 | 0.0001 |
| DME, C2H6O-1 | 46.07 | 0.0000 | 0.0000 | 0.0000 | 0.0000 | 0.0000 | 0.0000 | 0.0000 | 0.0000 | 0.9998 |
| Ethane, C2H6 | 30.07 | 0.0000 | 0.0000 | 0.0000 | 0.0000 | 0.0000 | 0.0000 | 0.0000 | 0.0000 | 0.0000 |
| Propane, C3H8 | 44.10 | 0.0000 | 0.0000 | 0.0000 | 0.0000 | 0.0000 | 0.0000 | 0.0000 | 0.0000 | 0.0000 |
| Acetic Acid, C2H4O-01 | 60.05 | 0.0000 | 0.0000 | 0.0000 | 0.0000 | 0.0000 | 0.0000 | 0.0000 | 0.0000 | 0.0000 |
| Acetone, C3H6O-01 | 58.08 | 0.0000 | 0.0000 | 0.0000 | 0.0000 | 0.0000 | 0.0000 | 0.0000 | 0.0000 | 0.0000 |
| Ethanol, C2H6O-02 | 46.07 | 0.0000 | 0.0000 | 0.0000 | 0.0000 | 0.0000 | 0.0000 | 0.0000 | 0.0001 | 0.0000 |
| Butanol, C4H10-01 | 74.12 | 0.0000 | 0.0000 | 0.0000 | 0.0000 | 0.0000 | 0.0000 | 0.0000 | 0.0000 | 0.0000 |
| Enthalpy Btu/lbmol | | −122360.00 | −22883.45 | −21810.37 | −7739.87 | −53229.89 | −27806.99 | −101950.00 | −117940.00 | −86847.89 |
| Enthalpy Btu/lb | | −6790.70 | −1879.55 | −1779.44 | −1869.70 | −2210.07 | −2153.13 | −3268.25 | −6544.70 | −1885.26 |
| Enthalpy MMBtu/hr | | −130340000.00 | −95529000.00 | −102390000.00 | −9011900.00 | −48687000.00 | −14453000.00 | −70263000.00 | −43105000.00 | −28114000.00 |
| Entropy Btu/lbmol-R | | −38.04 | −1.73 | −0.31 | −5.64 | −7.67 | −7.26 | −52.94 | −31.57 | −74.81 |
| Entropy Btu/lb-R | | −2.11 | −0.14 | −0.02 | −1.36 | −0.32 | −0.56 | −1.70 | −1.75 | −1.62 |

The H₂ rich stream removed from the H₂ membrane at 420 psig (30 bar) with a flow rate of 1,164.35 lbmol/hr has the following composition (Table 11).

TABLE 11
HYDROGEN RICH STREAM REMOVED
FROM THE HYDROGEN MEMBRANE

| PHASE | VAPOR |
|---|---|
| Temp., ° F. (° C.) | 125.0 (51.7) |
| Pressure, psig (bar) | 42.00 (30.0) |
| Flowrate, lbmol/hr | 1,164.35 |
| Composition | Mol % |
| CH₄ | 0.29 |
| CO₂ | 4.62 |
| N₂ | 0.23 |
| H₂O | 0.02 |
| CO | 0.27 |
| H₂ | 94.54 |
| CH₄O | 0.03 |

The H₂ rich stream removed from the H₂ membrane can either go through a PSA system to produce 7.50 MMSCFD of pure hydrogen at 400 psig (28.6 bar) or can be used as boiler fuel to produce 380 ton/day of 600 psig saturated steam for the catalytic distillation dehydration column reboiler and 6,503 HP of electric power which is about 80% of the power requirements for the entire DME plant.

Although the coupled purge rates is 80% and 5% in this example are quite different from that in Example 1i.e. 30% and 15%, the resulting inlet gases to the H₂ membrane system from both examples are quite similar both in gas compositions and flow rates (Table 12). It means that as long as the natural gas feed rate is kept constant, the same H₂ membrane system can be used for all cases when the ideal module number of 2.05 in the feed gases to the methanol synthesis loop is maintained.

TABLE 12
COMPARISON OF INLET GASES TO THE H₂ MEMBRANE
SYSTEM BETWEEN EXAMPLES 1 AND 3

| EXAMPLE | EXAMPLE 1 | EXAMPLE 3 |
|---|---|---|
| Purge Rate for the Methanol | 30 | 80 |
| Synthesis Loop | | |
| Purge Rate for Recycle Gas B | 15 | 5 |
| Inlet Gas Comp., mol % | | |
| CH₄ | 13.51 | 13.43 |
| CO₂ | 11.17 | 11.12 |
| N₂ | 2.88 | 7.03 |
| H₂O | 0.02 | 0.01 |
| CO | 11.80 | 8.95 |
| H₂ | 59.87 | 58.83 |
| CH₄O | 0.75 | 0.63 |
| TOTAL | 100.00 | 100.00 |
| Flow Rate, lbmol/hr | 2,097 | 2,079 |

The remaining recycle gas B (S2 in FIG. 5) contents a CH₄ flow of 261.98 lbmol/hr which accounts for 92.16% of the CH₄ slip in the steam reformer effluent (S9 in FIG. 5) and meanwhile enforces a 97.09% of CH₄ conversion for the natural gas feed stream to the saturator (S1 in FIG. 5). The results are summarized in Table 13.

TABLE 13
METHANE CONVERSION OF THE NATURAL GAS FEED
UNDER HIGH PRESSURE & MILD TEMPERATURE
FOR STEAM REFORMER OPERATION CONDITIONS

| Phase | Vapor |
|---|---|
| Steam Reformer Operating Pressure, psig (bar) | 300 (21.7) |
| Steam Reformer Operating Temperature, ° F. (° C.) | 1,600 (871) |
| CH₄ Conversion of the Natural Gas Feed, % | 97.09 |

| | | | Saturated Natural | |
|---|---|---|---|---|
| Component | Natural | Remaining | Gas & Remaining | Syngas |
| Molar Flow, | Gas to | Recycle Gas B | Recycle Gas B to | from |
| lbmol/hr | Separator | to Saturator | Reformer | Reformer |
| CH₄ | 766.15 | 261.98 | 1,026.64 | 284.25 |
| CO₂ | 5.63 | 168.48 | 171.23 | 299.94 |
| N₂ | 10.46 | 136.33 | 146.75 | 146.75 |
| H₂O | 0.00 | 0.01 | 2,003.23 | 1,084.81 |
| CO | 0.00 | 173.85 | 173.81 | 834.87 |
| H₂ | 0.00 | 116.18 | 116.12 | 2,589.21 |
| CH₄O | 0.00 | 12.10 | 0.06 | 0.00 |
| C₂H₆ | 20.12 | 0.00 | 20.07 | 0.02 |
| C₃H₈ | 2.42 | 0.00 | 2.41 | 0.00 |
| TOTAL | 804.78 | 868.93 | 3,660.32 | 5,239.85 |

When the natural gas feed stream is not combined with the remaining recycle gas B, then all the CH₄ slip in the steam reformer effluent will come from the natural gas feed stream and the CH₄ conversion of the natural feed stream to the saturator drops from 97.09% to 73.02% (Table 14).

TABLE 14
METHANE CONVERSION OF THE NATURAL GAS FEED
WHEN THE REMAINING RECYCLE GAS B IS NOT COMBINED
WITH THE NATURAL GAS FEED STREAM

| Phase | Vapor |
|---|---|
| Steam Reformer Operating Pressure, psig (bar) | 300 (21.7) |
| Steam Reformer Operating Temperature, ° F. (° C.) | 1,600 (871) |
| CH₄ Conversion of the Natural Gas Feed, % | 73.02 |

| | | Saturated Natural Gas | |
|---|---|---|---|
| Component Molar | Natural Gas to | & Remaining Recycle | Syngas from |
| Flow, lbmol/hr | Separator | Gas B to Reformer | Reformer |
| CH₄ | 766.15 | 765.43 | 206.53 |
| CO₂ | 5.63 | 4.25 | 153.45 |
| N₂ | 10.46 | 10.44 | 10.44 |
| H₂O | 0.00 | 1,516.31 | 760.80 |
| CO | 0.00 | 0.00 | 457.10 |
| H₂ | 0.00 | 0.00 | 1,943.21 |
| CH₄O | 0.00 | 0.00 | 0.00 |
| C₂H₆ | 20.12 | 20.10 | 0.01 |
| C₃H₈ | 2.42 | 2.41 | 0.00 |
| TOTAL | 804.78 | 2,318.94 | 3,531.54 |

In order to restore the high CH₄ conversion of the natural gas, the common practice of today's industrial applications is to increase the steam reformer operating temperature to 1,832° F. (1,000° C.) that improves the CH₄ conversion to 93.70%, and then reduces the steam reformer operating pressure to 200 psig (14.8 bar) that finally restores the CH₄ conversion to 97.09%. Of course, higher reformer operating temperature means higher fuel consumption; and lower syngas production pressure means higher syngas compressor compression power.

### Example 4

Keeping the same operating conditions as shown in Table 9, the MRF reactor is simulated by Aspen Plus Basic Engineering V7.3 using all the feed syngases in Table 1. The simulated results are summarized in Table 15 (Example 3 data are also included in the table for comparison purposes).

TABLE 15
SIMULATED MRF METHANOL REACTOR RESULTS USING
ALL THE FEED SYNGASES IN TABLE 1 UNDER THE SAME
OPERATING CONDITIONS AS SHOWN IN TABLE 9

| Methanol | Present | Present | | | | | |
|---|---|---|---|---|---|---|---|
| Synthesis | Invention | Invention | | | Exxon | | Johnson |
| Processes | 2 | 1 | UNITEL | ICI | Mobil | TEC | Matthey |

Feed Gas Comp., mol %

| | | | | | | | |
|---|---|---|---|---|---|---|---|
| CH₄ | 7.49 | 10.68 | 5.74 | 9.33 | 12.05 | 1.35* | 10.10 |
| CO | 18.76 | 15.75 | 9.08 | 8.70 | 10.31 | 7.90 | 4.89 |
| CO₂ | 7.65 | 9.50 | 10.60 | 10.45 | 4.14 | 5.80 | 3.27 |
| H₂ | 61.70 | 61.16 | 64.00 | 69.37 | 69.03 | 83.20 | 81.24 |
| H₂O | 0.42 | 0.22 | 0.24 | 0.11 | 0.10 | 0.10 | 0.12 |
| N₂ | 3.91 | 2.27 | 9.76 | 1.66 | 3.84 | 1.35* | 0.00 |
| CH₄O | 0.07 | 0.42 | 0.58 | 0.38 | 0.53 | 0.30 | 0.38 |
| TOTAL | 100.00 | 100.00 | 100.00 | 100.00 | 100.00 | 100.00 | 100.00 |

Outlet Gas Comp., mol %

| | | | | | | | |
|---|---|---|---|---|---|---|---|
| H₂ | 45.7 | 47.1 | 54.6 | 60.1 | 61.0 | 77.3 | 77.3 |
| CO | 6.9 | 5.9 | 3.7 | 3.3 | 3.0 | 2.2 | 1.4 |
| CO₂ | 10.0 | 11.2 | 10.2 | 9.6 | 3.7 | 4.4 | 1.9 |
| CH₄ + N₂ | 16.0 | 17.2 | 18.5 | 13.2 | 19.4 | 3.2 | 11.3 |
| H₂O | 1.3 | 1.7 | 2.7 | 3.2 | 1.4 | 2.7 | 1.9 |
| CH₄O | 20.1 | 16.9 | 10.3 | 10.6 | 11.5 | 10.2 | 6.2 |
| Total | 100.0 | 100.0 | 100.0 | 100.0 | 100.0 | 100.0 | 100.0 |
| Feed Gas Module | 2.05 | 2.05 | 2.71 | 3.08 | 4.49 | 5.65 | 9.56 |
| Number | | | | | | | |
| Feed Gas H₂/CO | 3.28 | 3.88 | 7.05 | 7.97 | 6.70 | 10.53 | 16.62 |
| Molar Ratio | | | | | | | |
| Feed Gas CO/CO₂ | 2.46 | 1.66 | 0.86 | 0.83 | 2.49 | 1.36 | 1.50 |
| Molar Ratio | | | | | | | |
| CH₄O Production | 14.36 | 12.32 | 10.31 | 8.44 | 8.94 | 8.23 | 5.21 |
| Based on 100 lbmol/hr | | | | | | | |
| Feed Gas, lbmol/hr | | | | | | | |
| H₂O Production Based | 1.26 | 1.05 | 2.08 | 2.52 | 1.06 | 2.18 | 1.57 |
| on 100 lbmol/hr Feed | | | | | | | |
| Gas, lbmol/hr | | | | | | | |
| CO Conversion, % | 74 | 72 | 66 | 68 | 76 | 77 | 75 |
| CO₂ Conversion, % | 6 | 11 | 20 | 24 | 26 | 38 | 48 |

*Assume equal amount of CH₄ and N₂ in the gas mixture.

Example 4 further illustrates the importance of having a module number in the feed gas to the methanol synthesis loop to be as close to 2.05 as possible. As shown in Table 15, a reduction of the module number from 5.65 (TEC) to 2.05 (Present Inventions) can increase the CH₄O production by 50% for Present Invention 1 or 74% for Present Invention 2; and even a slightly increase of the module number to 2.71 (UNITEL) can cause a loss in CH₄O production by 20% for Present Invention 1 or 39% for Present Invention 2.

### Example 5

Same as Example 3 except that the pressurized burner in the reformer is replaced by an atmospheric pressure burner. The primary heat transfer mechanism is radiation now rather than convection. A comparison of reformer process gas effluent temperatures, reformer flue gas effluent temperatures, reformer burner pressures, reformer fuel consumption, and reformer duties, etc. are shown in Table 16.

TABLE 16
A COMPARISON OF REFORMER PROCESS GAS EFFLUENT
TEMPERATURES, REFORMER FLUE GAS EFFLUENT
TEMPERATURES, REFORMER BURNER PRESSURES,
REFORMER FUEL CONSUMPTION, AND REFORMER
DUTIES, ETC. BETWEEN EXAMPLES 3 AND 5

| EXAMPLE | EXAMPLE 3 | EXAMPLE 5 |
|---|---|---|
| Reformer burner pressure, psig (bar) | 150 (11.4) | 2 (1.2) |
| Primary heat transfer | Convective | Radiative |
| Reformer process gas exit temperature, | 1,021 (549) | 1,600 (871) |
| ° F. (° C.) | | |
| Reformer flue gas exit temperature, | 1,080 (582) | 1,825 (996) |
| ° F. (° C.) | | |
| Reformer fuel (NG) consumption, | 284.05 (46%) | 615.40 (100%) |
| lbmol/hr | | |
| Reformer duty, MMBtu/hr | 79.17 (75%) | 105.71 (100%) |

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. The present invention provides a process for the production of DME comprising the following steps of:
Purging a portion of recycle gas from the H₂ membrane to the steam reformer HP burner;
Simultaneously subjecting a feedstock mixture including natural gas and the remaining of the recycle gas from the H₂ membrane to the steam reformer HP burner to the bottom of a saturator;
Feeding a hot water stream to the top of the saturator and allowing the hot water to evaporate in the presence of the rising gaseous stream as it travels down the saturator. In this way, all the high pressure steam required for the downstream steam reforming reactions is provided;
Steam reforming the saturated natural gas and the remaining recycle gas to produce a syngas;
Recovering the heat from the reformer effluent by superheating the saturated high pressure steam to generate electric power in a syngas heat recovery boiler and superheating boiler feed water to generate superheated medium pressure steam for additional electric power generation;
Directing the effluent from the medium pressure heat recovery boiler into a cooler where bulk of the water vapor in the syngas is condensed and knocked-out;
Combining the compressed syngas with the methanol synthesis loop recycle gas to yield a module number of 2.05 which is the ideal module number for methanol synthesis;
Subjecting the combined gas mixture to the methanol synthesis loop in the presence of Haldor Topsoe MK-121 methanol synthesis catalyst to obtain a reaction product gas mixture including methanol, carbon dioxide, water vapor, inerts like methane and nitrogen, and unconverted hydrogen and carbon monoxide;
Condensing the reaction product gas mixture to separate the methanol and the water produced;
Reducing the pressure of the crude methanol product to evaporate dissolved gases;
Purifying the low pressure crude methanol product by a light end distillation column to strip more dissolved gases;
Pumping the purified crude methanol product to a pressure of about 115 psig (9 bar) and then it is fed to a catalytic distillation dehydration column for the production of fuel grade DME.

2. The process as set forth in claim 1, wherein both the purge rate from the methanol synthesis loop to the H₂ membrane and the purge rate from the H₂ membrane to the pressurized reformer burner are manipulated to provide enough CO₂ in order to get 2.05 module number for the methanol synthesis feed gas and meanwhile also provide appropriate gas flow to evaporate enough steam in the saturator for the downstream steam reforming reactions.

3. The process as set forth in claim 1, wherein high purge rates of 25 to 85% for the methanol synthesis loop are required to keep the inert gases (CH₄ and N₂) and CO₂ at appropriate concentrations.

4. The process as set forth in claim 1, wherein purge rates of 2% to 20% for the recycle gas from the H₂ membrane to the saturator are also required to adjust the final concentrations of the inert gases and CO₂ in the feed gas to steam reformer. In general, a low purge rate from the methanol synthesis loop is coupled with a high purge rate for the recycle gas from the H₂ membrane to the saturator, and vice versa.

5. The process as set forth in claim 1, wherein higher CO₂ concentration in the methanol synthesis loop recycle gas gives higher molar heat capacity for the recycle gas stream and enables a lower recycle to make-up syngas molar ratio, such as 0.1 to 1.3 which improves the process economics.

6. The process as set forth in claim 1, wherein the crude methanol product stream is let down from methanol synthesis pressure to a pressure about 10 psig (1.7 bar) to release the dissolved gases first before it is fed to the light end distillation column. The purified crude methanol product from the light end distillation column is then pumped to the pressure required for the catalytic distillation dehydration column, mainly 116 psig (9 bar). By doing so, it saves about 80% of the condenser cooling duty and about 60% of the reboiler heat duty for the light end distillation column.

7. The process as set forth in claim 1, wherein no CO₂ adsorption system of any kind is required in the process of natural gas to DME via the methanol dehydration route.

8. The process as set forth in claim 1, wherein no external sources of CO₂ are used in the process to manipulate the module number for the feed gas to the methanol synthesis loop.

9. The process as set forth in claim 1, wherein the steam reformer HP burner can be replaced by a conventional low or atmospheric pressure burner.

10. The process as set forth in claim 1, wherein the hydrogen rich stream removed from the hydrogen membrane has a higher pressure than the HP burner fuel gas, it can be used to replace the natural gas feed to the HP burner without compression when electric power/steam generation is not desired.

11. The process as set forth in claim 10, wherein the hydrogen rich stream removed from the hydrogen membrane has a pressure between 300 to 420 psig, and pure hydrogen at 280 to 400 psig can be obtained through a PSA unit when electric power/steam generation is not desired.

12. The process as set forth in claim 1, wherein as long as the natural gas feed rate is fixed and a module number of 2.05 is maintained in the feed gases to the methanol synthesis loop in spite of the fact that huge differences in the coupled purge rates of the methanol synthesis loop and the recycle gas from the H₂ membrane to the saturator, the resulting inlet gases to the H₂ membrane system remain similar both in gas compositions and flow rates, and hence the same H₂ membrane system can be applied.

13. The process as set forth in claim 1, wherein the CH₄ content in the remaining recycle gas from the H₂ membrane to the saturator accounts for more than 90% of the CH₄ slip in the steam reformer effluent, which enforces a 96 to 98% CH₄ conversion of the natural gas feed stream to the saturator even at high steam reformer operation pressure (300 psig) and mild operation temperature (1,600° F.).

14. The process as set forth in claim 1, wherein the water stream produced at the catalytic distillation dehydration column bottom has a purity of 99.97 mol % or 99.94 wt %, and there is no need for any waste water treatment.

15. The process as set forth in claim 14, wherein the water stream produced at the catalytic distillation dehydration column bottom is combined with the knockout water and make-up boiler feed water and preheated by the methanol synthesis reactor effluents to provide all the hot water required for the saturator.
